(19) United States
(12) Patent Application Publication
(10) Pub. No.: US 2014/0128276 A1
(43) Pub. Date: May 8, 2014

(54) CYTOKINE PROFILES AS METHODS FOR DIAGNOSIS AND PROGNOSIS OF IRRITABLE BOWEL SYNDROME

(71) Applicants: Xuhang Li, Clarksville, MD (US); Philip Alex, Bel Air, MD (US); Michael Centola, Oklahoma City, OK (US); Nicholas Knowlton, Oklahoma City, OK (US)

(72) Inventors: Xuhang Li, Clarksville, MD (US); Philip Alex, Bel Air, MD (US); Michael Centola, Oklahoma City, OK (US); Nicholas Knowlton, Oklahoma City, OK (US)

(73) Assignees: OKLAHOMA MEDICAL RESEARCH FOUNDATION, Oklahoma City, OK (US); THE JOHNS HOPKINS UNIVERSITY, Baltimore, MD (US)

(21) Appl. No.: 13/996,019

(22) PCT Filed: Jan. 13, 2012
(86) PCT No.: PCT/US2012/021172

§ 371 Date: Jan. 17, 2014

Related U.S. Application Data
(60) Provisional application No. 61/432,358, filed on Jan. 13, 2011.

(51) Int. Cl. G01N 33/68

(52) U.S. Cl. CPC G01N 33/6863; G01N 33/6869
USPC 506/9; 436/501; 435/6.11; 435/7.92

(57) ABSTRACT

Methods, devices, and kits for diagnosing or evaluating irritable bowel syndrome employ an analysis for the presence and amount of specific cytokines. The levels of such cytokines provide an index for diagnosis and/or evaluation of therapeutic response. Samples to be tested include peripheral blood, serum, plasma, or tissue from a human subject having or suspected of having Irritable Bowel Syndrome.

### TECHNICAL FIELD OF THE INVENTION

This invention is related to the testing of cytokines for diagnostics, disease monitoring, and therapy monitoring. In particular, it relates to Irritable Bowel Syndrome.

### BACKGROUND OF THE INVENTION

Irritable bowel syndrome (IBS) is one of the most common functional gastrointestinal disorders, characterized by abdominal pain or discomfort/bloating and associated with disturbances in abnormal bowel function (diarrhea and/or constipation). Although only a minority of sufferers seek care for their symptoms, IBS accounts for annual direct costs of $8 billion and indirect costs of $25 billion in the United States, and an prevalence of 10-15% in N. America. Despite the prevalence and impact of IBS in the community, the pathophysiology of IBS is not fully understood, which in part is due to the fact that the etiology is not explainable by obvious local biochemical or structural causes. The disorder is now attributed to dysregulation of the brain-gut axis, with alterations at different levels and disturbed interplay of several factors, specifically the enteric, autonomic and/or central nervous systems, mucosal immune activation, an altered microbiotome, and psychosocial factors.

The therapeutic strategy for IBS has been focused on traditional therapies focused on individual symptoms, with limited efficacy in addressing the entire syndrome complex without any effects on the natural history of the disorder. The lack of definitive biomarkers has hampered diagnosis, prognosis, and the development of fully effective treatments.

The hallmark symptoms of IBS are similar to that of other gastrointestinal diseases, including Inflammatory Bowel Disease (IBD), which is characterized by chronic, progressive, systemic, autoimmune inflammatory disorder of the gastrointestinal tract. Some studies have also suggested that IBS is a type of low-grade IBD, and that these diseases may be inter-related. The diseases are however clinically distinct because IBS does not produce the destructive inflammation or intestinal bleeding found in IBD, or the harmful complications often occurring with IBD. Patients with IBS are not at higher risk for colon cancer, nor are they more likely to develop IBD or other gastrointestinal diseases. Furthermore, IBS seldom requires hospitalization, and treatment does not usually involve surgery or powerful medications, such as steroids or immunosuppressive agents. There is, however, similarity in early symptoms and signs of IBS with IBD, and given the lack of definitive biomarkers to distinguish between these two disorders, clinicians are often faced with difficult challenges to identify and distinguish between these two clinically distinct diseases.

Cytokines are defined as any of several regulatory proteins, such as the interleukins and lymphokines, that are released by cells of the immune system and act as intercellular mediators in the generation of an immune response. Cytokines are secreted by immune or other cells, whose action are on cells of the immune system, such as, but not limited to, T-cells, B-cells, NK cells and macrophages. Chemokines are defined as chemotactic cytokines produced by a variety of cell types in acute and chronic inflammation that mobilize and activate white blood cells. Cytokines and chemokines are important cell signaling proteins, mediating a wide range of physiological and pathological responses, including immunity, inflammation, and hematopoiesis.

Several therapeutic agents, primarily directed at cytokines, are currently available and have shown great promise in the treatment of various inflammatory conditions of the bowel. While previous studies have evaluated systemic cytokine profiles in IBS, they have been limited to a relatively small number of cytokines and the analysis of absolute level of each cytokine, without consideration of the interplay of multiple cytokines. There are no tests available for determining whether patients have a specific cytokine antagonized by a therapeutic agent, or whether patients will positively respond to such medications. Furthermore, despite the need to identify cytokine associations with IBS, there have been no definitive link identified between cytokine levels and diagnosis, prognosis, and treatment response in such pathologic states. There is a continuing need in the art better to identify and distinguish IBS and to monitor the course of the disease.

### SUMMARY OF THE INVENTION

According to one aspect of the invention a method is provided to aid in diagnosing Irritable Bowel Syndrome (IBS). A patient sample is tested to determine level of one or more cytokines selected from the group consisting of IL-5, IL-6, IL-8, IL-12, IL-13, IL-15, IL-17, and CCL-2. The patient level is compared to a reference range of levels determined in healthy subjects. A level in the patient that falls outside of the reference range is identified as indicating IBS.

Another aspect of the invention is a method to aid in distinguishing between IBS and Irritable Bowel Disease (IBD). A patient sample is tested to determine level of one or more cytokines selected from the group consisting of IL-6, IL-10, IL-12, TNF-α, and CCL-2. The patient level is compared to a reference range of levels determined for IBS patients and to a reference range of levels determined for IBD subjects. A level in the patient that falls within the IBS reference range is identified as indicating IBS and a level in the patient that falls within the IBD range is identified as indicating IBD.

Still another aspect of the invention is a method to monitor response to a therapy for IBS in a patient receiving therapy. A patient sample is tested to determine level of one or more cytokines selected from the group consisting of IL-1β, IL-6, IL-12, TNF-α, and CCL-2. The level is compared to a reference level previously determined in the patient prior to therapy or at a previous time point during therapy. A change in the level compared to the reference is identified as indicating responsiveness to the therapy.

According to one aspect of the invention a method is provided to aid in diagnosing Irritable Bowel Syndrome (IBS). A patient sample is tested to determine level of one or more mRNA molecules encoding a cytokine selected from the group consisting of IL-5, IL-6, IL-8, IL-12, IL-13, IL-15, IL-17, and CCL-2. The patient level is compared to a reference range of levels determined in healthy subjects. A level in the patient that falls outside of the reference range is identified as indicating IBS.

Another aspect of the invention is a method to aid in distinguishing between IBS and Irritable Bowel Disease (IBD). A patient sample is tested to determine level of one or more mRNA molecules encoding a cytokine selected from the group consisting of IL-6, IL-10, IL-12, TNF-α, and CCL-2. The patient level is compared to a reference range of levels determined for IBS patients and to a reference range of levels determined for IBD subjects. A level in the patient that falls within the IBS reference range is identified as indicating IBS and a level in the patient that falls within the IBD range is identified as indicating IBD.

Still another aspect of the invention is a method to monitor response to a therapy for IBS in a patient receiving therapy. A patient sample is tested to determine level of one or more mRNA molecules encoding a cytokine selected from the group consisting of IL-1β, IL-6, IL-12, TNF-α, and CCL-2. The level is compared to a reference level previously determined in the patient prior to therapy or at a previous time point during therapy. A change in the level compared to the reference is identified as indicating responsiveness to the therapy.

Another aspect of the invention is a kit for diagnosing Irritable Bowel Syndrome (IBS). In a divided or undivided container are five or more antibodies which specifically bind to a distinct cytokine selected from the group consisting of IL-5, IL-6, IL-8, IL-12, IL-13, IL-15, IL-17, and CCL-2.

Still another aspect of the invention is a kit for distinguishing between Irritable Bowel Syndrome (IBS) and Irritable Bowel Disease (IBD). In a divided or undivided container are three or more antibodies which specifically bind to a distinct cytokine selected from the group consisting of IL-6, IL-10, IL-12, TNF-α, and CCL-2.

Yet another aspect of the invention is a kit for monitoring response to a therapy for Irritable Bowel Syndrome (IBS). In a divided or undivided container are three or more antibodies which specifically bind to a distinct cytokine selected from the group consisting of IL-1β, IL-6, IL-12, TNF-α, and CCL-2.

A further aspect of the invention is a device to aid in diagnosing Irritable Bowel Syndrome (IBS). It comprises five or more antibodies which specifically bind to a distinct cytokine selected from the group consisting of IL-5, IL-6, IL-8, IL-12, IL-13, IL-15, IL-17, and CCL-2, and a means of detection of antibody binding to a sample component.

Still another aspect of the invention is a device to aid in distinguishing between Irritable Bowel Syndrome (IBS) and Irritable Bowel Disease (IBD). It comprises three or more antibodies which specifically bind to a distinct cytokine selected from the group consisting of IL-6, IL-10, IL-12, TNF-α, and CCL-2; and a means of detection of antibody binding to a sample component.

Another aspect of the invention is a device to aid in monitoring response to a therapy for Irritable Bowel Syndrome (IBS). The device comprises three or more antibodies which specifically bind to a distinct cytokine selected from the group consisting of IL-1β, IL-6, IL-12, TNF-α, and CCL-2; and a means of detection of antibody binding to a sample component.

These and other embodiments which will be apparent to those of skill in the art upon reading the specification provide the art with methods, kits, and devices for diagnosing or monitoring or predicting that an individual has or will develop IBS.

### BRIEF DESCRIPTION OF THE DRAWINGS

FIG. 1 depicts the pair-wise comparison of the unique cytokine profiles from serum of IBS patients and unaffected healthy controls. Serum levels of cytokines were assessed using biometric immunosandwich ELISAs. A broad sensitivity range of standards ranging from 1.95-32000 pg/ml were used to help enable the quantitation of a wide dynamic range of cytokine concentrations while still providing high sensitivity. Cytokines significantly elevated in IBS patients relative to controls are denoted with an asterisk.

FIG. 2 depicts the validation of the observed systemic cytokine profiles from tissues of patients with IBS, relative to healthy controls. Colonic biopsies from both IBS and controls were obtained after informed consent. Immunocytochemical detection was determined in 4 μm paraffin-embedded sections from these biopsies, heat fixed and endogenous peroxidase blocked with 0.3% H2O2 in methanol. Following washes and preblocking, tissue sections were incubated overnight (4° C.) with antibodies to IL-6, IL-12, and TNF-α. Sections were washed twice again, incubated with Alexa Fluor IgG antibodies, following which they were washed, incubated with 1% Sudan Black, and treated with Hoescht 33342. Tissue sections were finally mounted, sealed, and examined using an Zeiss 510 Meta confocal microscope (Zeiss, Maple Grove, Minn.).

FIG. 3 depicts the discriminative potential of the cytokine profiles from IBS, from those of patients with diarrhea predominant disease and those with constipation predominant disease, and relative to unaffected healthy controls. A forward step-wise multivariate discriminant functional analysis was used to select a set of analytes that maximally discriminate among subgroups of IBS and controls. The discriminatory potential of the final equation was observed as a line plot of the root values obtained for each group.

FIG. 4 provides a visual plot of the discriminative potential of the cytokine profiles from IBS, relative to that of IBD (Ulcerative colitis and Crohn's Disease) and unaffected healthy controls.

FIG. 5 depicts the changes in serum cytokine profiles with relevance to clinical response of IBS patients treated with placebo effects. Treatment strategies, included placebo acupuncture, and placebo acupuncture augmented with a patient-practitioner relationship. Serum cytokines were measured in patients prior to and for two visits following treatment.

FIG. 6 depicts the changes in cytokine profiles of therapeutic responders in serum cytokine profiles with relevance to the absence of clinical response (i.e., non-response) inof IBS patients treated with clinical placebo effects,. Treatment strategies, included placebo acupuncture, and/or placebo acupuncture augmented with a patient-practitioner relationship. Serum cytokines were measured in patients prior to treatment and for two visits following.

FIGS. 7A-7B. FIG. 7A represents the multidimensional scaling analysis (MDS) that was utilized to generate dimensions that can interpret statistically significant differences between cytokine networks in IBS patients treated with placebo effects. Strong positive clusters between sets of cytokines identified in treated, responsive IBS patients are depicted in FIG. 7A. The clusters identified were in striking contrast to those of baseline. Baseline levels (FIG. 7B), indicate the intricate but distinct immune network associated with IBS diagnostics and prognostics.

### DETAILED DESCRIPTION OF THE INVENTION

The inventors have developed methods of diagnosing, predicting, monitoring an individual who has or who will develop IBS. The methods use selected sets of cytokines which are distinctive for comparisons with normal unaffected individuals and with individuals who have similar but different disease pathology. Sets of cytokines can also be followed over time to see changes in the disease state of an individual with IBS.

Patient samples which may be analyzed in the methods include peripheral blood, serum, plasma, tissue samples, or other body fluid sample, such as stool, CSF, tears, saliva, and lymph. The patient may have mild, intermediate, or severe disease symptoms. Predefined levels for normal unaffected individuals, for affected IBS individuals, and for affected IBD individuals may include a median level or a range of levels of the cytokine or cytokine set found in such samples. The samples may be from a healthy subject, a diseased patient, or a patient having associated symptoms of IBS, and/or extra-intestinal involvement. Typically samples will be collected in a clinic and transferred to a laboratory for analysis. Once results are obtained, they may be transmitted back to the clinic or to the individual patient. Such transmission may be by any means, including electronic, oral, telephonic, or written. Results may be transmitted as raw data, as processed data, and/or as a conclusion.

The terms “indicates” or “correlates” in reference to a parameter, e.g., a modulated proportion, level, or cellular localization in a sample from a patient, may mean that the patient has IBS. The term “comparing” refers to making an assessment of how the proportion, level or cellular localization of one or more cytokines in a sample from a patient relates to the proportion, level or cellular localization of the corresponding one or more cytokines in a standard or control sample. The parameter may comprise the presence, absence and/or particular amounts of one or more cytokines. In other embodiments a parameter may comprise a weight in a multivariate algorithm (e.g. BOOSTED models, C&RT, Random Forests, Penalized regression models). The term “pattern” may mean a multivariate algorithm. A particular set or pattern of one or more cytokines (including the presence, absence, and/or particular amounts) may indicate that a patient has IBS (or correlates to a patient having IBS).

Different sets of cytokines have been identified which appear to be optimum for a particular type of determination. For example, where the disease state to be determined is IBS relative to healthy unaffected individuals, the cytokine set comprises one or more cytokines selected from the group consisting of interleukin IL-5, IL-6, IL-8, IL-12, IL-13, IL-15, IL-17, and CCL-2 (MCP-1). Any whole number of these cytokines can be assessed, from 1 to 8, inclusive. Typically a single index is derived from the cytokine levels of the set. Where the disease states to be distinguished are IBS and IBD, the cytokines of the set can be selected from the group consisting of interleukin IL-6, IL-10, IL-12, TNF-α, and CCL-2(MCP-1). Any whole number of these cytokines from 1 to 5, inclusive, can be tested and the data combined into a single index. Where the disease state is IBS, and therapeutic monitoring is desired, the cytokine set can be selected from the group consisting of interleukin IL-1β, IL-6, IL-12, TNF-α, and CCL-2 (MCP-1). Any whole number of these cytokines from 1 to 5 may be used, inclusive, and the data combined into a single index. Various algorithms may be used to provide the single index. Optionally, the single index reflecting levels of cytokine expression may be combined with other clinical assessments, for example, pain, constipation, or diarrhea.

Antibodies can conveniently, but not exclusively, be used in characterizing the cytokine contents of various patient samples. Exemplary techniques which can be used include Enzyme Linked Immunosorbent Assay (ELISA) and its derivatives. Additionally, the antibody can be used in immunoblot or Western blot analysis (WB). The antibodies of the present invention may also be used in conjunction with fresh-frozen and/or formalin-fixed, paraffin-embedded tissue blocks, such as blocks prepared from a tumor biopsy, prepared for study by immunohistochemistry (IHC). Another form of immunoassay involves protein array technology, which allows high-throughput screening. Typically these employ an array of antibodies for specifically capturing proteins. Non-antibody based assays which can be used for cytokines are bioassays which test for a cytokine's effect on particular cells. The cytokine biomarkers may optionally be detected by mass spectrometry or alternatively by means of an electrochemical luminescent assay. Other detection means that can be employed include optical methods, electrochemical methods (voltametry and amperometry techniques), atomic force microscopy, and radio frequency methods.

Methods for analyzing levels of RNA typically are performed by specific hybridization, either to the RNA itself or to its reverse transcription product, cDNA. Specific probes may be used which are complementary to the RNA or to one or both strands of the cDNA. The sequences of the cytokine encoding genes are known in the art and can be used to design probes. Probes can be used in liquid or solid phase hybridization, such as on a nucleic acid array or on microparticles. Such methods are well known in the art.

Kits which can be formulated specifically to practice the methods of the invention may contain separate antibodies or nucleic acid probes or arrays or other aggregate or composite reagents. The kits may include other reagents for running the assays, including for example, secondary antibodies, labels, chromogenic reagents, chromophores, solid phases for binding or for separating binding products or cytokines prior to binding, including gels, chromatography matrices, buffers, enzymes, etc. Instructions for running assays including standard values (medians, ranges, etc.) for normal or diseased patients may also be provided as part of the kit. Because the kits are specifically formulated for the purposes of using the sets of cytokines disclosed here, they will not contain probes for the entire proteome or the entire transcriptome. Rather they will contain antibodies or probes for less than 100, less than 75, less than 50, less than 40, less than 30, less than 20, less than 15, or less than 10, less than 8 or less than 5 cytokines or cytokine encoding sequences.

Devices can be used for running the analyses as disclosed for the selected sets of cytokines. The devices may contain an array of antibodies or nucleic acid probes. The devices may contain detection means for identifying and quantitating binding of an analyte to the antibody or probe. The detection means may be any known in the art suitable for detecting fluorescence, radioactivity, color, heat, etc. Optionally, but desirably, the device will contain software for combining the results of levels of the determined cytokines or cytokine expression. Optionally, but desirably, the device will contain software for comparing the results between test samples and control samples. Optionally, the device will contain an output means for providing results, such as electronically, on paper, audibly, etc.

The power of a diagnostic test to correctly predict status is measured as the sensitivity or specificity of the assay or the area under a receiver operated characteristic (“ROC”) curve. The cytokine panels of the present invention may show a statistical difference in different IBS statuses of at least p<0.05, p<10-2, p<10-3, p<10-4 or p<10-5. Diagnostic tests that use these cytokines may show an ROC of at least 0.6, at least 0.7, at least 0.8, at least 0.9. The values measured for markers of a cytokine panel may be mathematically combined and the combined value correlated to the underlying diagnostic question. Advanced multivariate analyses including those of cluster analysis, factor analysis, discriminant function analysis (DFA), and multidimensional scaling were used to provide detailed characterization of cytokine-based IBS disease profiles and can be used to make diagnoses and evaluations. Cytokine measurements may be compared with relevant diagnostic amounts, cut-offs, ranges, or multivariate model scores that distinguish a positive IBS status from a negative IBS status.

Complementary multivariate analytical methods provide a vivid picture of the biological significance of the immune profile network. A DFA is distinct from the above analyses in that it is a class distinction modeling method that identifies sets of variables that best discriminate predefined disease and treatment groups. Multidimensional scaling provides a means of identifying correlational configurations of statistically significant cytokines, and allows for a visual representation of the pattern of proximities within the groups studied. These methods and analytical tools were utilized to identify novel diagnostic discriminatory cytokine biomarkers that can be used to distinguish sufficiently one IBS disease subtype from each other and controls. Furthermore, these tools were utilized to develop diagnostic, prognostic, disease activity-based, predictive, and therapeutic response panel of markers in patients with IBS disease subtypes.

Multidimensional scaling is an iterative process to detect meaningful underlying dimensions to explain observed similarities or dissimilarities between the groups studied [Borg I, Groenen P J F. Modern Multidimensional Scaling: Theory and Applications (Springer Series in Statistics). Springer; 2005]. This analysis uses correlational matrices to construct configurations of the data in a lower dimensional matrix, such that the relative distances between the groups are similar to those in the higher dimensional matrix. The degree of correspondence between the distances and the matrix input by the user is measured (inversely) by a stress function defined by Phi=Σ[dij−f(δij)]2, where dij stands for the euclidean distance, and δij stands for the observed distance. The proximities and distances are then represented on a two-dimensional Shepard diagram scatterplot which facilitates visualization and the interpretation of patterns. All statistical analyses for MDS were performed with R software [Ihaka R, Gentleman R. R: A Language for Data Analysis and Graphics. Journal of Computational and Graphical Statistics. 1996; 5:299-314].

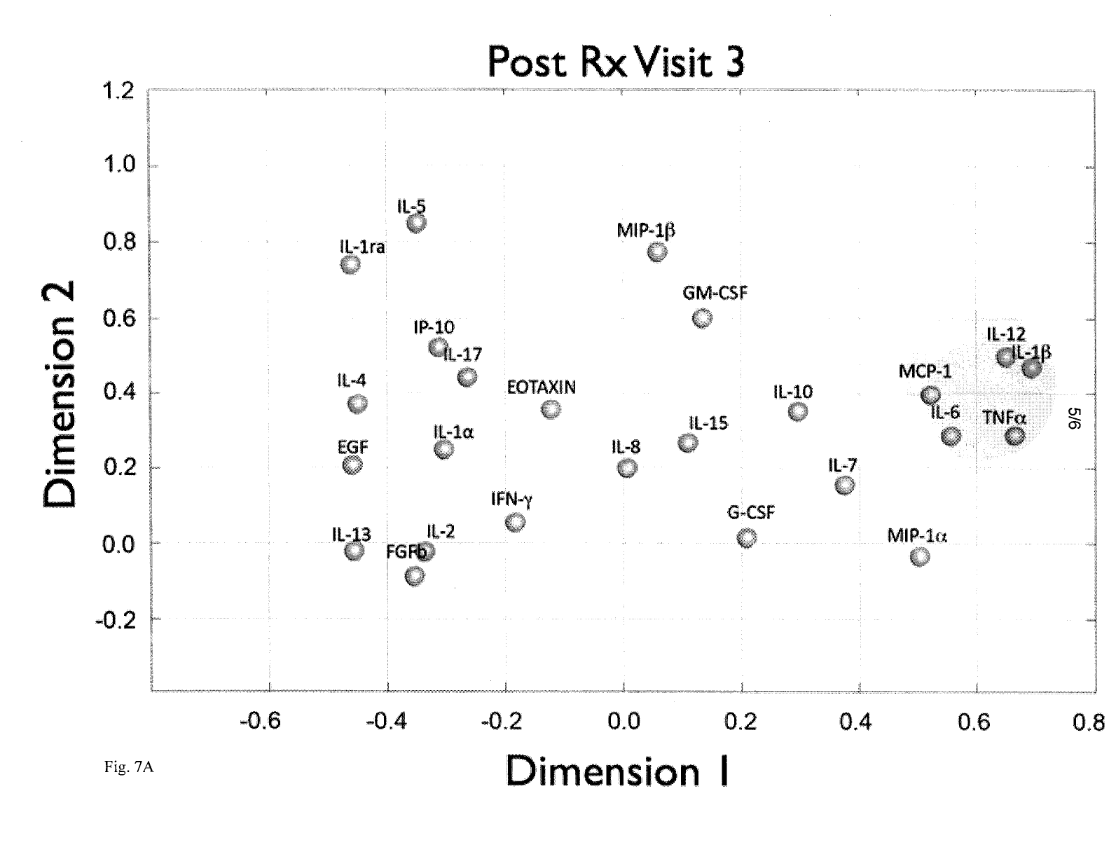

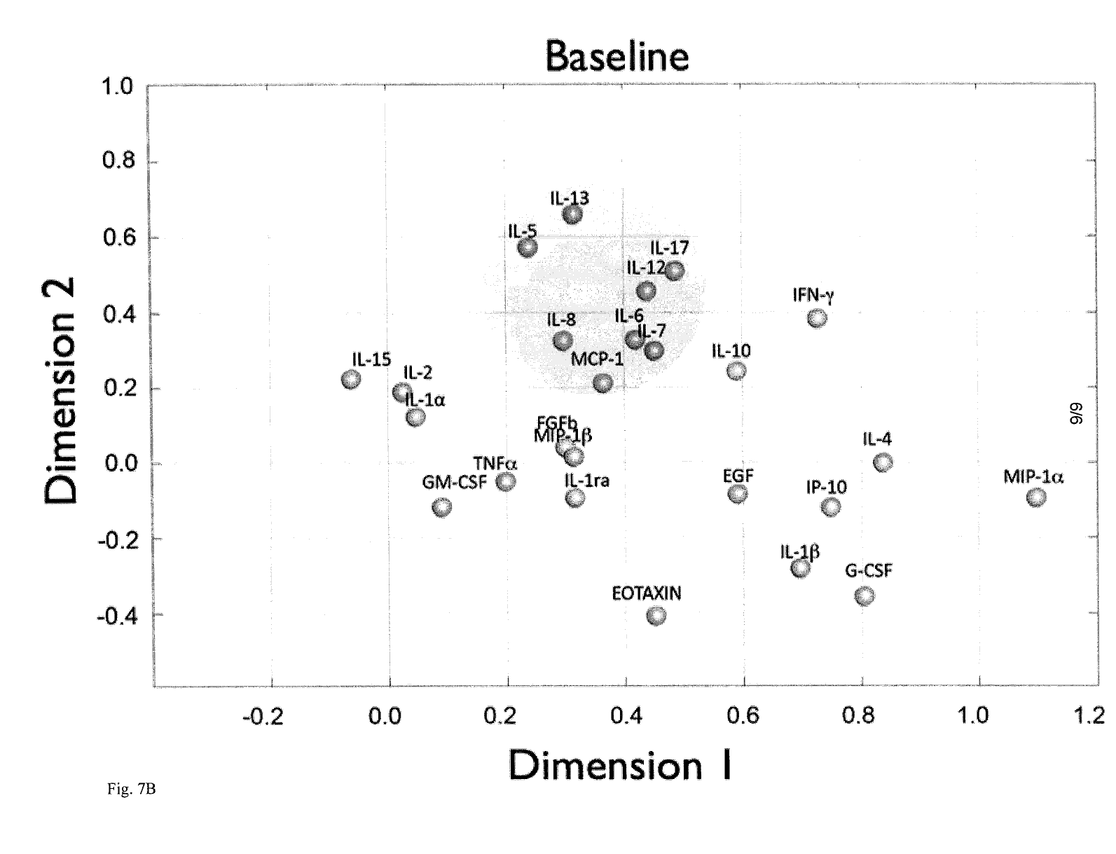

As depicted in FIGS. 7A-7B, Multidimensional Scaling Analysis of the cytokine patterns in IBS patients in a clinical trial with placebo effects identified a strong positive cluster between IL-12, and IL-1β (r=0.705, p=0.036), and between IL-6, TNFα, and MCP-1 (r=0.819, p=0.014) in treated patients at Visit 3 (FIG. 7A), whereas these clusters were significantly absent for the same patients at Baseline. At baseline, the multidimensional scaling analysis of cytokine patterns also showed a significantly positive correlation between IL-6, IL-7, IL-8, IL-12, IL-17, and MCP-1 (r=0.692, p=0.030), whereas those clusters were absent post treatment (FIG. 7B). These unique representations provide a visual inspection of similarities and differences between cytokine changes among the groups, indicating the intricate but distinct immune network associated with IBS diagnostics and prognostics.

Discriminant Function Analysis (DFA) is a multivariate class distinction algorithm that allows one to construct a mathematical model of discrimination built in a stepwise manner. This analysis was used here to identify the cytokines that best discriminated between patients with IBS and controls, and was modeled as previously described [(Alex P, Szodoray P, Knowlton N et al. Multiplex serum cytokine monitoring as a prognostic tool in rheumatoid arthritis. Clin Exp Rheumatol. 2007; 25:584-592.), (Szodoray P, Alex P, Jonsson M V et al. Distinct profiles of Sjogren's syndrome patients with ectopic salivary gland germinal centers revealed by serum cytokines and BAFF. Clin Immunol. 2005; 117:168-176.), (Jarvis J N, Dozmorov I, Jiang K et al. Novel approaches to gene expression analysis of active polyarticular juvenile rheumatoid arthritis. Arthritis Res Ther. 2004; 6:R15-R32.)]. Specifically, at each step, all variables are reviewed to determine which will maximally discriminate among groups. These variables are then included in a discriminative function, denoted a root, which is an equation consisting of a linear combination of cytokine changes used for the prediction of group membership. Variables will continue to be included in the model, as long as the respective F values for those variables are larger than the standard threshold (established by the analytical package Statistica, StatSoft, Tulsa, Okla., USA). The discriminant potential of the final equation from the forward stepwise DFA can then be observed in a simple multidimensional plot of the values of the roots obtained for each group. This multivariate approach identifies groups of analytes, the changes of which in levels can delineate profiles and create diagnostic patterns.

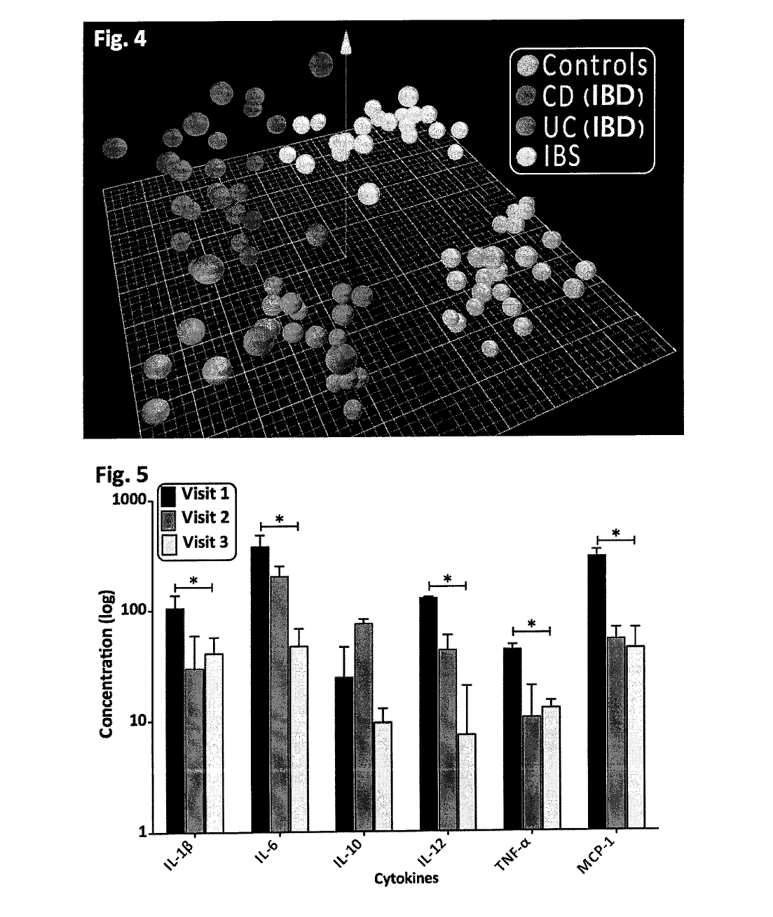

The utility of this analysis is that it identifies groups of analytes, the changes of levels in which can delineate profiles and create diagnostic patterns in IBS. Results of DFA can be visualized on a multidimensional plot, with class discrimination power represented by distance between both subtypes of IBS. All disease states were readily distinguished from controls (FIG. 4). This was then included in a discriminative function, denoted a root, which is an equation consisting of a linear combination of changes in analytes used for the prediction of group membership, and as shown in FIG. 3 was able to discriminate IBS from unaffected controls. To further evaluate the potential of the analysis to discriminate IBS from IBD (our prior published IBD data), we further added analysis from the cytokine levels from serum of IBD patients into this pool. An F test was used to determine the statistical significance of the discriminatory power of the selected analytes, which was also characterized by a Wilk's Lambda coefficient. This coefficient ranges from 1.0 (no discriminatory power) to 0.0 (perfect discriminatory power), and was able to identify six cytokines IL-6, IL-10, IL-12, TNF-α, and CCL-2 with the power to discriminate IBD from IBS, and unaffected controls. The discriminant potential of the final equation from the forward stepwise DFA could then be observed in a simple multidimensional plot of the values of the roots obtained for each group as represented in FIG. 4. This multivariate approach further validated the distinctive cytokine profiles, of which the changes in levels can delineate profiles and create diagnostic patterns.

To develop an index of cytokine levels using the results of these multivariate analyses that could be readily interpreted in a clinical context for the medical community, a scoring system was created, denoted the IBS Activity Index (IBSAI). These values represent the levels deduced from an algorithm containing an aggregate of relevant cytokine measurements where the root values from the discriminant functional analysis were normalized such that the maximal value of controls was 0. The normal IBSAI range was calculated in a standard manner, e.g., normal range=the 25-75% interquartile range of unaffected control values.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLES

We assayed for inflammatory mediators in patients with IBS, and evaluated their role of these mediators in the immunopathogenesis of the disease. The results provide a vivid understanding of the mechanisms and mediators involved in the GI tract in IBS, and generate profiles that can enable effective diagnosis of IBS. Cytokine profiles that were identified at the systemic level were also validated locally in colonic tissues. We used multivariate approaches that further identified patterns that can uniquely distinguish IBS from IBD, which is a common differential diagnosis for IBS. We further evaluated the potential of these cytokines in therapeutic prognosis for IBS, and were able to identify profiles that characterize both response and non-response for patients with IBS. While some individual cytokines may have previously been associated with the syndrome, we found multi-cytokine patterns which can be used to diagnose, correlate with disease activity, and facilitate prognosis in IBS.

### Example 1

We performed multiplex serum cytokine profiling from serum of IBS patients and controls. The cohort also included unaffected age and sex matched unaffected controls. The following 24 cytokines were assessed: IL-1ra, IL-1α, IL-β, IL-2, IL-4, IL-5, IL-6, IL-10, IL-12, IL-13, IL-17, IFN-γ, TNF-α, G-CSF, GM-CSF, IL-8, MIP-1α, MIP-1β, MCP-1 (also known as CCL-2), EGF, VEGF, FGF-basic, IP-10, and Eotaxin. (Invitrogen)

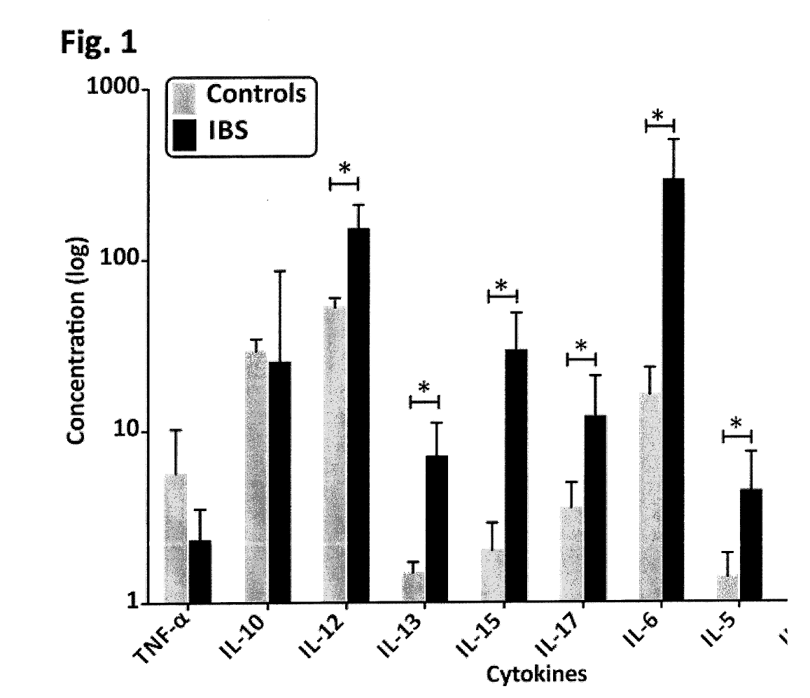

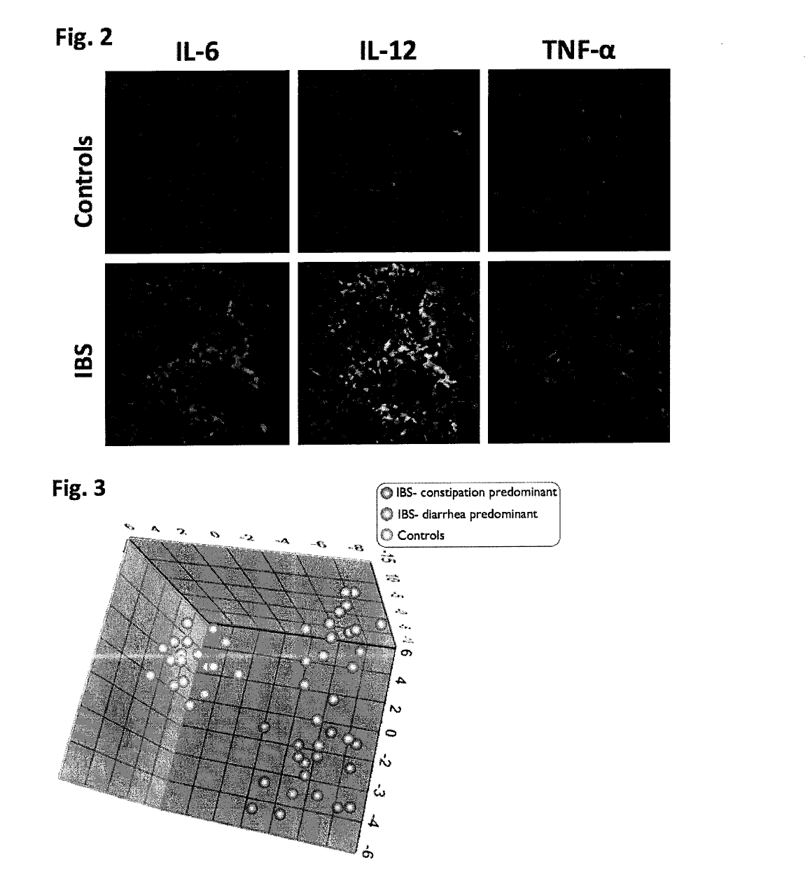

As shown in FIG. 1, when compared to unaffected controls, IBS patients demonstrated significantly elevated levels of (IL)-5, IL-6, IL-8, IL-12, IL-13, IL-15, IL-17, and CCL-2, suggesting a Th1-Th2-chemotactic profile in IBS. Such a profile demonstrates their significance in immunomodulatory pathogenesis and indicates their importance as serological biomarkers for IBS. This was further validated at the tissue level using biopsies from patients with IBS relative to unaffected controls (FIG. 2).

### Example 2

To evaluate the potential of the cytokine profiles to discriminate IBS from that of unaffected controls, a multivariate analysis called discriminant functional analysis was used for selection of the set of analytes that maximally discriminate among IBS and controls built in a step-wise manner. This was then included in a discriminative function, denoted a root, which is an equation consisting of a linear combination of changes in analytes used for the prediction of group membership, and as shown in FIG. 3 was able to discriminate IBS from unaffected controls. To evaluate further the potential of the analysis to discriminate IBS from IBD (our prior published IBD data), we further added analysis from the cytokine levels from serum of IBD patients into this pool. An F test was used to determine the statistical significance of the discriminatory power of the selected analytes, which was also characterized by a Wilk's Lambda coefficient. This coefficient ranges from 1.0 (no discriminatory power) to 0.0 (perfect discriminatory power), and as shown in FIG. 4 we were able to identify six cytokines (IL-6, IL-10, IL-12, TNF-α, and CCL-2) with the power to discriminate IBD from IBS, and from unaffected controls.

### Example 3

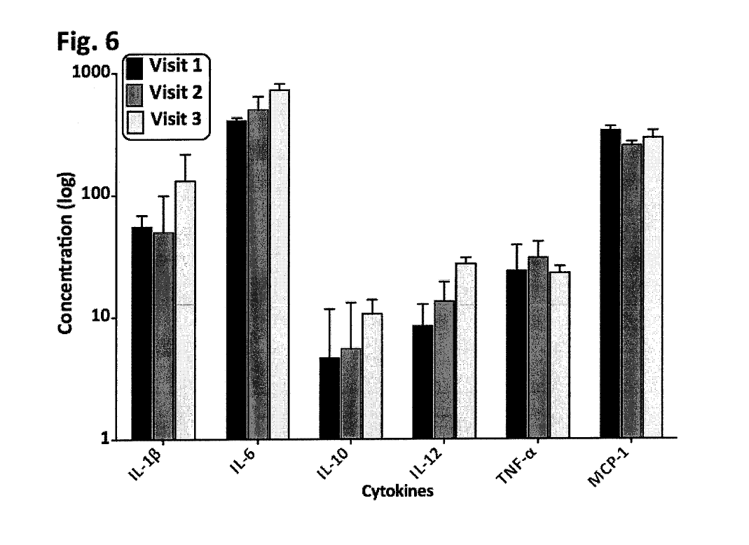

To investigate whether treatment with personalized approaches may play a role in the effective mediation of the identified immunomodulatory profiles, IBS patients in a clinical trial treatment with clinical placebo effects (placebo acupuncture alone, or placebo acupuncture with an augmented patient-practitioner relationship) were followed through three clinical visits. Responders and non-responders were identified based on clinical indices including global improvement scale (range 1-7), adequate relief of symptoms, symptom severity score, and quality of life. When the discriminatory cytokine profiles were correlated with clinical scores, they were able to significantly differentiate response from non-response as shown in FIG. 5 (for response), and FIG. 6 (for non-response), suggestive of the profound prognostic potential of the cytokine profiles in IBS.

### REFERENCES

The disclosure of each reference cited is expressly incorporated herein.
1. Chen et al., “Identification of novel serological biomarkers for Inflammatory Bowel Disease using Escherichia coli proteome chip,” Molecular & Cellular Proteomics 8:1765-1776, 2009
2. Li et al., “An old herbal medicine with a potentially new therapeutic application in inflammatory bowel disease,” Int. J. Clin. Exp. Med. 4:309-319, 2011
3. Li et al., “New serological biomarkers of inflammatory bowel disease,” World J. Gastroeneterol 14:5115-5124, 2008
4. Axel, et al., “Applications of proteomics in the study of Inflammatory Bowel Disease: Current status and future directions with available technologies,” Inflamm Bowel Dis 15:616-629, 2009

1. A method to aid in diagnosing Irritable Bowel Syndrome (IBS), comprising the steps of:
testing a patient sample to determine level of one or more cytokines or level of one or more mRNA molecules encoding a cytokine, wherein said cytokine is selected from the group consisting of IL-5, IL-6, IL-8, IL-12, IL-13, IL-15, IL-17, and CCL-2;
comparing the patient level to a reference range of levels determined in healthy subjects;
identifying a level in the patient that falls outside of the reference range as indicating IBS.

2. A method to aid in distinguishing between IBS and Irritable Bowel Disease (IBD), comprising:
testing a patient sample to determine level of one or more cytokines or level of one or more mRNA molecules encoding a cytokine, wherein said cytokine is selected from the group consisting of IL-6, IL-10, IL-12, TNF-α, and CCL-2;
comparing the patient level to a reference range of levels determined for IBS patients and to a reference range of levels determined for IBD subjects;
identifying a level in the patient that falls within the IBS reference range as indicating IBS and identifying a level in the patient that falls within the IBD range as indicating IBD.

3. A method to monitor response to a therapy for IBS in a patient receiving therapy, comprising:
testing a patient sample to determine level of one or more cytokine or level of one or more mRNA molecules encoding a cytokine, wherein said cytokine is selected from the group consisting of IL-1β, IL-6, IL-12, TNF-α, and CCL-2;
comparing the level to a reference level previously determined in the patient prior to therapy or at a previous time point during therapy;
identifying a change in the level compared to the reference as indicating responsiveness to the therapy.

4. The method of claim 1 wherein the patient sample is selected from the group consisting of a tissue sample, a serum sample, a blood sample, a saliva sample, a urine sample, a stool sample and a cerebrospinal fluid (CSF) sample.

5. The method of claim 2 wherein the level of five cytokines is determined and combined to form a single value representative of the patient's condition.

6. The method of claim 3 wherein the cytokine level is determined by an antibody-based detection technique.

7. The method of claim 6 wherein the technique is selected from the group consisting of immunoblotting, immunohistochemistry, immunoprecipitation, radioimmunosassay, ELISA, and antibody array binding.

8. The method of claim 1 wherein the patient level is further compared to a reference range of levels determined for an IBS subtype, wherein a patient level that falls within a subtype reference range is indicative of the patient having that subtype of IBS.

9. The method of claim 8 wherein the IBS subtype is constipation-dominant IBS.

10. The method of claim 8 wherein the IBS subtype is diarrhea-dominant IBS.

11. The method of claim 1 wherein the level is used to diagnose in combination with clinical evaluations.

12. The method of claim 2 wherein the level is used to distinguish in combination with clinical evaluations.

13. (canceled)

14. (canceled)

15. (canceled)

16. The method of claim 1 wherein mRNA levels are determined by reverse transcription of mRNA and measurement of cDNA levels.

17. A kit for diagnosing Irritable Bowel Syndrome (IBS), distinguishing between Irritable Bowel Syndrome (IBS) and Irritable Bowel Disease (IBD), or for monitoring response to a therapy for Irritable Bowel Syndrome (IBS), comprising in a divided or undivided container: (a) five or more antibodies which specifically bind to a distinct cytokine selected from the group consisting of IL-5, IL-6, IL-8, IL-12, IL-13, IL-15, IL-17, and CCL-2; (b) three or more antibodies which specifically bind to a distinct cytokine selected from the group consisting of IL-6, IL-10, IL-12, TNF-α, and CCL-2; or (c) three or more antibodies which specifically bind to a distinct cytokine selected from the group consisting of IL-1β, IL-6, IL-12, TNF-α, and CCL-2.

18. The kit of claim 17 which comprises six or seven of said antibodies of (a); or four or five of said antibodies of (b) or (c).

19. (canceled)

20. (canceled)

21. (canceled)

22. (canceled)

23. (canceled)

24. (canceled)

25. (canceled)

26. A device to aid in diagnosing Irritable Bowel Syndrome (IBS), distinguishing between Irritable Bowel Syndrome (IBS) and Irritable Bowel Disease (IBD), or for monitoring response to a therapy for Irritable Bowel Syndrome (IBS), comprising: (a) five or more antibodies which specifically bind to a distinct cytokine selected from the group consisting of IL-5, IL-6, IL-8, IL-12, IL-13, IL-15, IL-17, and CCL-2; (b) three or more antibodies which specifically bind to a distinct cytokine selected from the group consisting of IL-6, IL-10, IL-12, TNF-α, and CCL-2; or (c) three or more antibodies which specifically bind to a distinct cytokine selected from the group consisting of IL-1β, IL-6, IL-12, TNF-α, and CCL-2, and
a means of detection of antibody binding to a sample component.

27. The device of claim 26 which comprises six or seven of said antibodies of (a); or four or five of said antibodies of (b) or (c).

28. (canceled)

29. (canceled)

30. (canceled)

31. (canceled)

32. (canceled)

33. (canceled)

34. (canceled)
